(19) United States
(12) Patent Application Publication
(10) Pub. No.: US 2010/0296624 A1
(43) Pub. Date: Nov. 25, 2010

(54) HIGH-FREQUENCY SADDLE-TRAJECTORY FOR AXIAL CARDIAC CT

(75) Inventors: Claas Bontus, Hamburg (DE); Roland Proksa, Hamburg (DE); Thomas Koehler, Norderstedt (DE)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/445,765

(22) PCT Filed: Oct. 16, 2007
(86) PCT No.: PCT/IB07/54211

§ 371 Date: Apr. 16, 2009

(30) Foreign Application Priority Data
Oct. 18, 2006 (EP) 06122516.5

(51) Int. Cl. A61B 6/03

(52) U.S. Cl. 378/4

(57) ABSTRACT

If, in cardiac CT, the time window becomes shorter than the time required for a complete rotation of the gantry, the volume that can be reconstructed becomes small due to the non-existence of related pi-lines. According to an exemplary embodiment of the present invention, an examination apparatus is provided which generates a radiation beam oscillating in z-direction with an oscillation frequency higher than the rotational frequency of the source. This may provide for an exact image reconstruction of large volumes.

The invention relates to the field of tomographic imaging. In particular, the invention relates to an examination apparatus for examination of an object of interest, to an X-ray tube, a method of examination of an object of interest, a computer-readable medium and a program element.

The saddle-trajectory is one of the measures, which have been proposed to improve axial scanning with future large-area detectors. A saddle-trajectory is very attractive because it may result in a complete data set. For each object-point within a rather large volume at least two pi-lines exist. The existence of pi-lines is important, because pi-lines are related to the back-projection interval of exact reconstruction methods.

Some properties of a large class of saddle-trajectories were already discussed in J. D. Pack et al., Phys. Med. Biol., 49 (11) (2004) 2317, which is hereby incorporated by reference. J. D. Pack et al. showed that at least two pi-lines exist for object-points within a rather large volume. An exact filtered back-projection reconstruction algorithm was recently published by H. Yang et al., Phys. Med. Biol., 51 (5) (2006) 1157, which is hereby incorporated by reference.

Besides the technical feasibility and the question whether a sufficient image space is reconstructable, it is also important to evaluate the trajectory for clinical applications. One of the most interesting applications for the saddle-trajectory is cardiac CT. This application demands a high temporal resolution at a pre-defined phase-point in a cardiac cycle, which relates to a projection data interval. Exact reconstruction algorithms, which make use of the existence of pi-lines, are typically short scan or even supershort scan methods. However, the back-projection interval cannot be selected freely, but is determined by a pi-line. For the standard saddle, this may result in a strong spatial variation of the effective phase-point.

For cardiac CT, typically a cardiac time window is selected, which determines the data available for the reconstruction. The window should be small for a good temporal resolution. If the window becomes shorter than the time required for a complete rotation of the gantry, the volume that can be reconstructed by an exact method becomes small due to the non-existence of related pi-lines.

It would be desirable to provide for an improved focal spot trajectory enabling an exact image reconstruction in cardiac CT.

According to an exemplary embodiment of the present invention, an examination apparatus for examination of an object of interest is provided, the examination apparatus comprising a radiation source adapted for emitting an electromagnetic radiation beam with a focal spot to the object of interest and for rotating around a z-axis around the object of interest with a rotational frequency, wherein the radiation source is further adapted such that the focal spot of the beam moves back and forth along the z-direction of the z-axis with an oscillation frequency which is higher than the rotational frequency of the source.

Therefore, the examination apparatus may be adapted for performing a reciprocating focal spot movement during rotation of the radiation source. The reciprocating focal spot may be such that an exact volume image reconstruction becomes possible without additionally moving the object of interest (which may be a patient).

Thus, the examination apparatus comprises a radiation source which enables a high frequency movement of the focal spot along the patient axis during gantry rotation. In this way, cone-beam artefacts due to image reconstruction may be avoided.

According to another exemplary embodiment of the present invention, the oscillation frequency is more than two times higher than the rotational frequency of the radiation source. According to another exemplary embodiment of the present invention, the oscillation frequency is about six times higher than the rotational frequency.

This may provide for a rather homogeneous distribution of the pi-lines for object-points within such a high-frequency saddle-trajectory.

According to another exemplary embodiment of the present invention, the focal spot of the beam moves on a saddle-trajectory during rotation of the radiation source around the z-axis.

Furthermore, according to another exemplary embodiment of the present invention, the focal spot of the beam moves on a triangular trajectory during rotation of the radiation source around the z-axis.

According to another exemplary embodiment of the present invention, the radiation source or a part of the radiation source is adapted for performing a movement along the z-direction in order to move the focal spot along the z-direction.

Therefore, according to this exemplary embodiment of the present invention, a purely mechanical movement of the radiation source or of certain elements of the radiation source may provide for the reciprocating focal spot movement along the z-direction.

According to another exemplary embodiment of the present invention, the radiation source comprises an electron source for generating an electron beam and an anode, wherein, for moving the focal spot along the z-direction, the radiation source is adapted for deflecting the electron beam before it impinges on the anode.

Thus, no mechanical movement may be necessary for providing the focal spot movement along the z-direction. Such an electron beam deflection may be realized by the provision of deflection elements adapted for deflecting the electron beam by electric forces.

According to another exemplary embodiment of the present invention, the anode is adapted as a rotating anode with a focal track for moving the focal spot along the z-direction, wherein the rotating anode is adapted for rotating around a rotational axis and wherein the focal track has one of a sinusoidal form, a triangular form, and a saddle shaped form.

Therefore, the examination apparatus may be adapted for performing a reciprocating focal spot movement by simply rotating the anode of the X-ray tube.

According to another exemplary embodiment of the present invention, a detector for a detection of electromagnetic radiation from the radiation source and a reconstruction unit for a reconstruction of an image of the object of interest on the basis of the detected radiation are comprised within the examination apparatus.

The detector, according to a further exemplary embodiment of the present invention, is adapted for moving along the z-direction in the opposite direction of the source.

Therefore, the image quality may be further improved.

According to another exemplary embodiment of the present invention, the detected radiation comprises oscillation data resulting from the back and forth movement of the focal spot and, in combination, circular data resulting from a circular trajectory of the focal spot.

Furthermore, according to another exemplary embodiment of the present invention, the oscillation data is detected during a first rotation of the radiation source, wherein the circular data is detected during a second rotation of the radiation source.

Thus, the circular and the oscillation data are detected during different rotations of the source.

According to another exemplary embodiment of the present invention, the radiation source is adapted as a dual-tube system, wherein the oscillation data and the circular data are detected simultaneously.

This may provide for a further reduction of measurement time.

According to another exemplary embodiment of the present invention, the examination apparatus is configured as one of the group consisting of a material testing apparatus, a medical application apparatus and a micro CT system.

A field of application of the invention may be medical imaging, in particular cardiac CT.

According to another exemplary embodiment of the present invention, the examination apparatus is adapted as one of a three-dimensional computer tomography apparatus and a three-dimensional rotational X-ray apparatus.

It should be noted in this context, that the present invention is not limited to computer tomography, but may always then be applied when the focal spot of an X-ray beam has to move in a reciprocating or oscillating manner.

Furthermore, according to another exemplary embodiment of the present invention, the electromagnetic radiation beam is adapted as a polychromatic X-ray beam.

According to another exemplary embodiment of the present invention, an X-ray tube for an examination apparatus is provided, which is adapted for emitting an electromagnetic radiation beam with a focal spot to an object of interest to be examined and for rotating around a z-axis around the object of interest with a rotational frequency, wherein the X-ray tube is further adapted such that the focal spot of the beam moves back and forth along the z-direction of the z-axis with an oscillation frequency which is higher than the rotational frequency of the source.

Furthermore, according to another exemplary embodiment of the present invention, a method of examination of an object of interest with an examination apparatus is provided, the method comprising the steps of emitting, by a radiation source, an electromagnetic radiation beam with a focal spot to the object of interest, rotating of the source around a z-axis around the object of interest with a rotational frequency, and moving of the focal spot of the beam back and forth along the z-direction of the z-axis with an oscillation frequency which is higher than the rotational frequency of the source.

This may provide for a focal spot trajectory generation enabling an exact image reconstruction for an increased volume of interest without having to move the object of interest back and forth.

According to another exemplary embodiment of the present invention, a computer-readable medium is provided, in which a computer program for examination of an object of interest is stored which, when being executed by a processor, causes the processor to carry out the above-mentioned method steps.

Furthermore, according to another exemplary embodiment of the present invention, a program element for examination of an object of interest is provided, which, when being executed by a processor, causes the processor to carry out the above-mentioned method steps.

It should be noted, that the X-ray tube with the z-moving focus may also comprise a collimation unit (or aperture system) between the output window of the tube and the object of interest, and that this collimation unit is capable of adapting very fast to the different z-positions of the focus, such that for every position of the focus the entire object of interest may be projected onto the detector by means of the X-ray beam. This fast adaptable collimator may for example have moving parts, or may be adapted in the form of a plurality of fixed collimation slits.

The method of examination of the object of interest may be embodied as the computer program, i.e. by software, or may be embodied using one or more special electronic optimization circuits, i.e. in hardware, or the method may be embodied in hybrid form, i.e., by means of software components and hardware components.

The program element according to an embodiment of the invention is preferably loaded into working memories of a data processor. The data processor may thus be equipped to carry out embodiments of the methods of the present invention. The computer program may be written in any suitable programming language, such as, for example, C++ and may be stored on a computer-readable medium, such as a CD-ROM. Also, the computer program may be available from a network, such as the WorldWideWeb, from which it may be downloaded into image processing units or processors, or any suitable computers.

It may be seen as the gist of an exemplary embodiment of the present invention that an examination apparatus is provided with a radiation source generating a focal spot reciprocated along the patient axis during rotation of the source. Since the oscillation frequency of the focal spot is higher than the rotational frequency of the source, a plurality of pi-lines exist, which are distributed rather homogeneously for object-points within such a high frequency trajectory. This may provide for an exact reconstruction even for large volumes.

These and other aspects of the present invention will become apparent from and elucidated with reference to the embodiments described hereinafter.

Exemplary embodiments of the present invention will be described in the following, with reference to the following drawings.

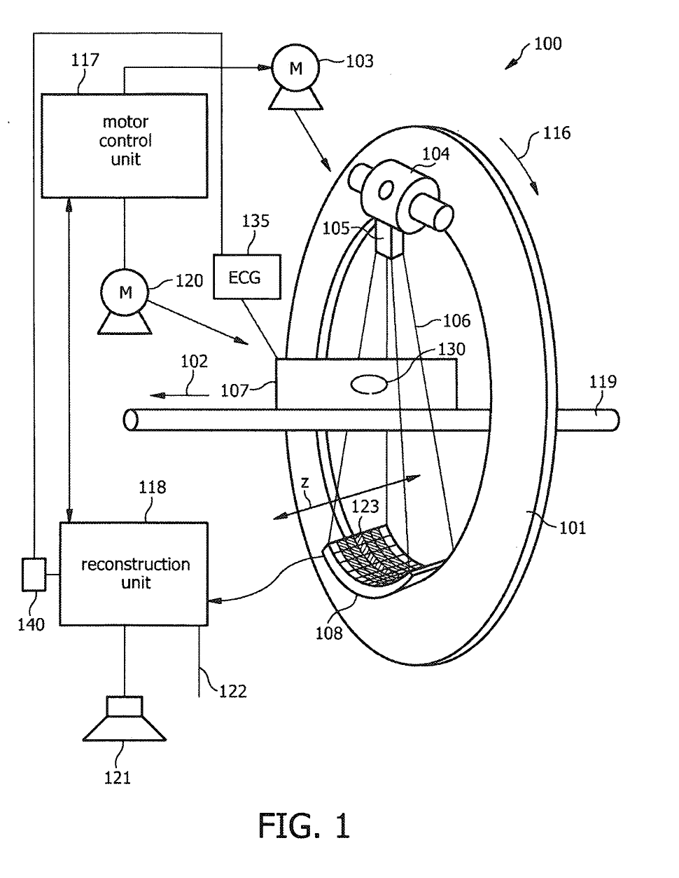

FIG. 1 shows a simplified schematic representation of an examination apparatus according to an exemplary embodiment of the present invention.

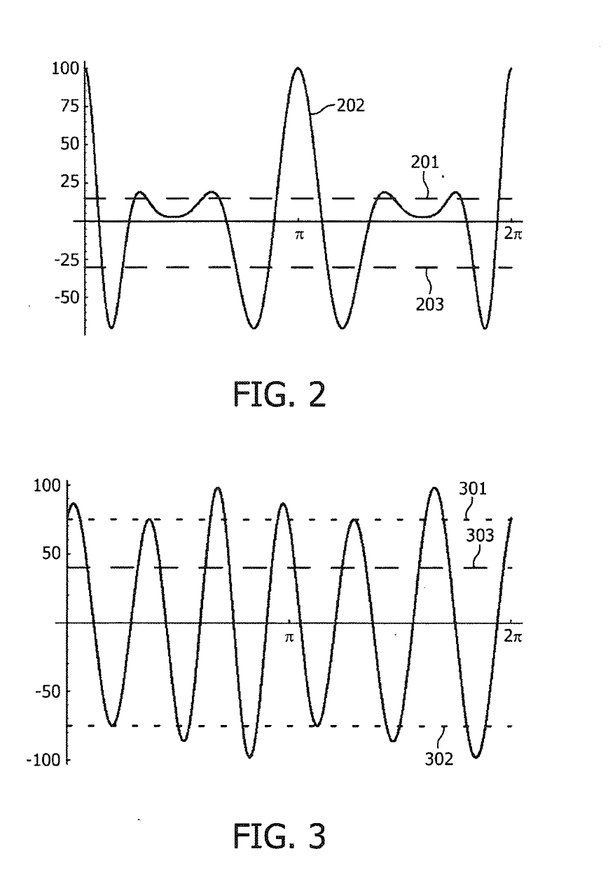

FIG. 2 shows a pi-curve according to an exemplary embodiment of the present invention.

FIG. 3 shows a pi-curve according to another exemplary embodiment of the present invention.

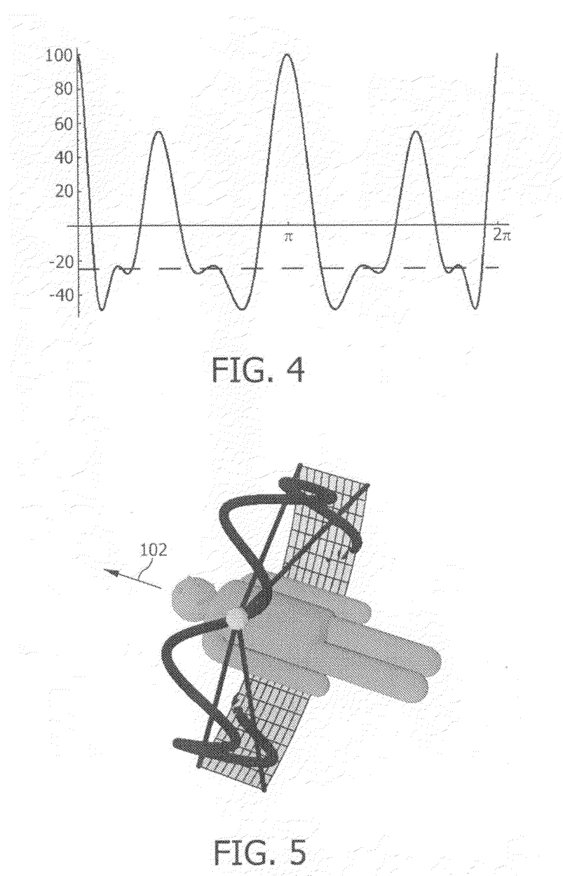

FIG. 4 shows a pi-curve according to another exemplary embodiment of the present invention.

FIG. 5 shows a schematic representation of a high-frequency saddle-trajectory according to an exemplary embodiment of the present invention.

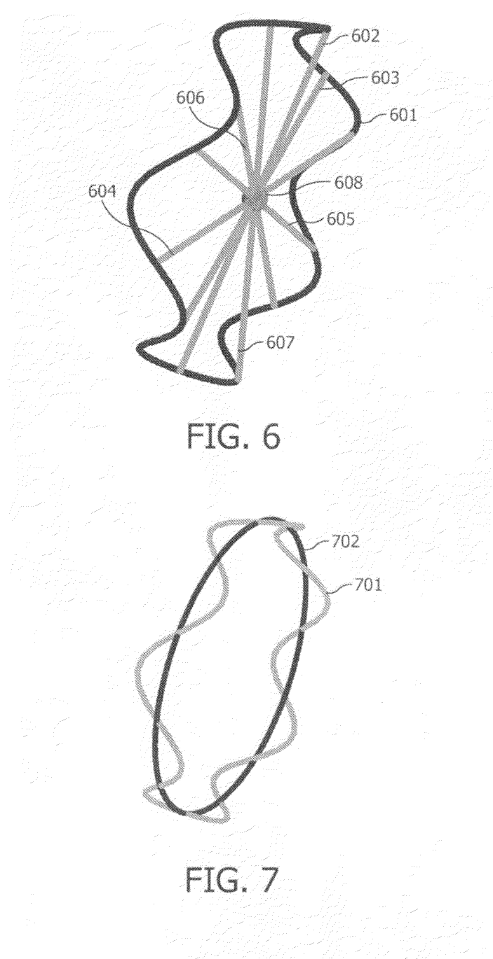

FIG. 6 shows pi-lines of an object-point for such a high-frequency saddle-trajectory.

FIG. 7 shows a saddle-trajectory combined with a circular trajectory according to an exemplary embodiment of the present invention.

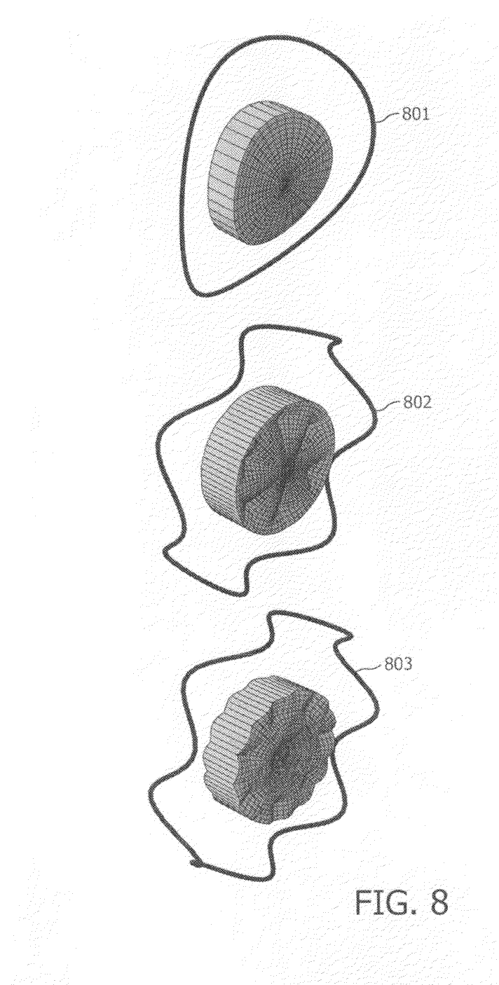

FIG. 8 shows a volume of object-points, for which pi-lines exist, for three different oscillations frequencies.

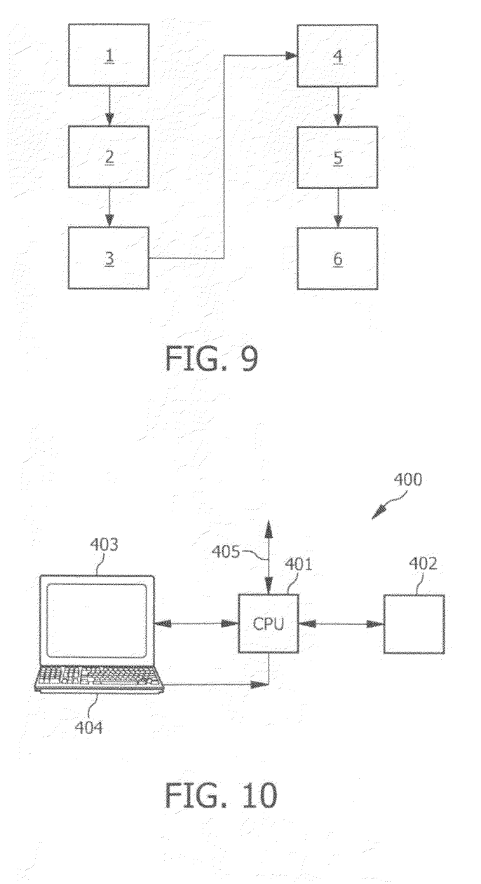

FIG. 9 shows a flow-chart of an exemplary method according to the present invention.

FIG. 10 shows an exemplary embodiment of an image processing device according to the present invention for executing an exemplary embodiment of a method in accordance with the present invention.

The illustration in the drawings is schematic. In different drawings, similar or identical elements are provided with the same reference numerals.

FIG. 1 shows an exemplary embodiment of a computed tomography scanner system according to the present invention. The computed tomography apparatus 100 depicted in FIG. 1 is a cone-beam CT scanner. The CT scanner comprises a gantry 101, which is rotatable around a rotational axis 102. The gantry 101 is driven by means of a motor 103. Reference numeral 104 designates a source of radiation such as an X-ray source, which, according to an aspect of the present invention, emits polychromatic or monochromatic radiation and comprises an X-ray tube.

Reference numeral 105 designates an aperture system which forms the radiation beam emitted from the radiation source 104 to a cone-shaped radiation beam 106. The cone-beam 106 is directed such that it penetrates an object of interest 107 arranged in the centre of the gantry 101, i.e. in an examination region of the CT scanner, and impinges onto the detector 108. As may be taken from FIG. 1, the detector 108 is arranged on the gantry 101 opposite to the source of radiation 104, such that the surface of the detector 108 is covered by the cone-beam 106. The detector 108 depicted in FIG. 1 comprises a plurality of detector elements 123 each capable of detecting X-rays which have been scattered by or passed through the object of interest 107.

During scanning the object of interest 107, the source of radiation 104, the aperture system 105 and the detector 108 are rotated along the gantry 101 in the direction indicated by arrow 116. For rotation of the gantry 101 the source of radiation 104, the aperture system 105 and the detector 108, the motor 103 is connected to a motor control unit 117, which is connected to a reconstruction unit 118.

The object of interest 107 may be, for example, a patient which is disposed on an operation table 119. During the scan of, e.g., the heart 130 of the patient 107, the gantry 101 rotates around the patient 107 and the focal spot moves along a saddle-trajectory. Therefore, a circular scan is performed without displacement of the operation table 119 parallel to the rotational axis 102.

Moreover, an electrocardiogram device 135 may be provided which measures an electrocardiogram of the heart 130 of the patient 107 while X-rays attenuated by passing the heart 130 are detected by detector 108. The data related to the measured electrocardiogram are then transmitted to the reconstruction unit 118.

The detector 108 is connected to the reconstruction unit 118. The reconstruction unit 118 receives the detection result, i.e. the read-outs from the detector elements 123 of the detector 108 and determines a scanning result on the basis of these read-outs. Furthermore, the reconstruction unit 118 communicates with the motor control unit 117 in order to coordinate the movement of the gantry 101 with motors 103 and 120 with the operation table 119.

The reconstruction 118 may be adapted for reconstructing an image from read-outs of the detector 108. A reconstructed image generated by the reconstruction unit 118 may be output to a display (not shown in FIG. 1) via an interface 122. The reconstruction unit 118 may be realized by a data processor to process read-outs from the detector elements 123 of the detector 108.

The measured data, namely the cardiac computer tomography data and the electrocardiogram data are processed by the reconstruction unit 118 which may be further controlled via a graphical user-interface 140.

It should be noted, however, that the present invention is not limited to this specific data acquisition reconstruction.

FIG. 2 shows a pi-curve according to an exemplary embodiment of the present invention. The horizontal axis shows the rotational angle of the source and the vertical axis shows the distance of a specific Pi-line to the xy-plane at specific coordinates (x,y). (as in

FIGS. 3 and 4).

The saddle-trajectory which is considered here may, for example, be located on a cylinder surface with harmonic oscillations in the z-direction. Generally, such a saddle may be parameterized as

y(s)=(R cos(s),R sin(s),z₀ cos (fs)),

with f≧2. Here, R corresponds to the cylinder radius, z₀ corresponds to the amplitude of the z-oscillations, and s is an angular variable. In the following, the values for f are restricted to integer values and any saddle with f>2 is denoted as high-frequency saddle. However, it should be noted that f may also be a non-integer value.

A pi-line is a line, which contains two points of the trajectory. For every object-point on a pi-line, exact reconstruction is in principle possible.

In the following, any coordinates (x,y) are fixed and the line parallel to the z-axis is considered as containing point (x,y,0). For each point on the trajectory y(s) there is a unique pi-line intersecting with this specified line at a certain z=z_{I}. In FIG. 2, z_{I}(s) is plotted for sε[0,2π] with (x,y)=(150, 0), f=6. Curves corresponding to the curve depicted in FIG. 2 are denoted as pi-curves.

As an example, point (x,y,z)=(150, 0, 15) is considered, which corresponds to the upper dashed line in FIG. 2. The dashed line 201 has 12 intersection points with the pi-curve 202. In other words, six pi-lines can be found for the object-point (x,y,z)=(150,0,15), since each pi-line intersects twice with the trajectory. The lower dashed line 203 in FIG. 2 corresponds to point (x,y,z)=(150, 0, −30). Obviously, for this point only four pi-lines exist. The number of object-points, which have six pi-lines increases for smaller distances to the rotation axis.

FIG. 3 illustrates this for (x,y)=(−50, 50). Again, as in FIGS. 2, f=6, R=570 and z₀=100.

Six pi-lines can be found for all object-points in the z-range between the two dotted lines 301, 302 in FIG. 3. For some object-points even eight pi-lines exist for f=6, as illustrated in FIG. 4. The dashed line in FIG. 4 corresponds to (x,y,z)=(200, 0, −25). f=6, R=570, z₀=100.

FIG. 5 shows a high-frequency saddle-trajectory for which the focal spot oscillates in the z-direction 102, while the tube-detector system rotates around the patient.

FIG. 6 shows the pi-lines 602-607 of an object-point 608 which are distributed homogeneously for such a high-frequency saddle-trajectory 601. The object-point 608 depicted in FIG. 6 is located at (x,y,z)=(−50, 50, 40), which corresponds to the dashed line 303 in FIG. 3.

As already mentioned, such a high-frequency saddle 601 may be realized by a movement of the tube or parts of the tube in the z-direction. Alternatively or additionally, a tube may be used, in which only the electron beam is deflected, for example by a plurality of deflection elements.

FIG. 7 shows a saddle-trajectory 701 which is combined with a circular trajectory 702. Here, the circular data may be obtained during other rotations using the same tube as for the saddle-trajectory. The circular data may also be obtained simultaneously with the saddle data using a dual-tube system.

FIG. 8 shows the volume of object-points for which pi-lines exist with respect to different focal spot trajectories 801, 802, 803. The three images in FIG. 8 correspond to f=2,6,7, respectively. It should be noted that for the case f=7 no pi-lines exist for a large number of object-points close to the rotation axis.

The concept of pi-lines may only be useful, if the object-points are illuminated over the complete back-projection interval.

A further improvement for fixed z₀ may be obtained, if a detector movement is realized also in the z-direction in opposite direction to the source.

FIG. 9 shows a flow-chart of an exemplary method according to the present invention for examination of an object of interest. The method starts at step 1 with the emission of an electron beam from a cathode towards an anode. Then, in step 2, the electron beam hits the anode, thus generating an electromagnetic radiation beam with a focal spot, which is directed towards the object of interest.

Then, in step 3, the X-ray tube is rotated around a z-axis around the object of interest. Then, in step 4, the focal spot of the X-ray beam is moved back and forth along the z-direction with an oscillation frequency which is higher than the rotational frequency of the X-ray tube.

In step 5, the X-rays which have passed the object of interest are detected. Simultaneously, ECG data are detected by an electrocardiogram. In a final step 6, an image of the heart is reconstructed according to an exact reconstruction scheme.

FIG. 10 depicts an exemplary embodiment of a data processing device 400 according to the present invention for executing an exemplary embodiment of a method in accordance with the present invention.

The data processing device 400 depicted in FIG. 10 comprises a central processing unit (CPU) or image processor 401 connected to a memory 402 for storing an image depicting an object of interest, such as a patient or an item of baggage. The data processor 401 may be connected to a plurality of input/output network or diagnosis devices, such as a CT device. The data processor 401 may furthermore be connected to a display device 403, for example, a computer monitor, for displaying information or an image computed or adapted in the data processor 401. An operator or user may interact with the data processor 401 via a keyboard 404 and/or other output devices, which are not depicted in FIG. 10.

Furthermore, via the bus system 405, it may also be possible to connect the image processing and control processor 401 to, for example, a motion monitor, which monitors a motion of the object of interest. In case, for example, a lung of a patient is imaged, the motion sensor may be an exhalation sensor. In case the heart is imaged, the motion sensor may be an electrocardiogram.

Exemplary embodiments of the invention may be sold as a software option to CT scanner console, imaging workstations or PACS workstations.

It should be noted that the term “comprising” does not exclude other elements or steps and the “a” or “an” does not exclude a plurality. Also elements described in association with different embodiments may be combined.

It should also be noted that reference signs in the claims shall not be construed as limiting the scope of the claims.

1. An examination apparatus for examination of an object of interest, the examination apparatus comprising:
a radiation source adapted for emitting an electromagnetic radiation beam with a focal spot to the object of interest and for rotating around a z-axis around the object of interest with a rotational frequency;
the radiation source being further adapted such that the focal spot of the beam moves back and forth along the z-direction of the z-axis with an oscillation frequency which is higher than the rotational frequency of the source.

2. The examination apparatus of claim 1,
wherein the oscillation frequency is about six times higher than the rotational frequency.

3. The examination apparatus of claim 1,
wherein the focal spot of the beam moves on a saddle trajectory during rotation of the radiation source around the z-axis.

4. The examination apparatus of claim 1,
wherein the focal spot of the beam moves on a triangular trajectory during rotation of the radiation source around the z-axis.

5. The examination apparatus of claim 1,
wherein, for moving the focal spot along the z-direction, the radiation source or a part of the radiation source is adapted for performing a movement along the z-direction.

6. The examination apparatus of claim 1,
wherein the radiation source comprises an electron source for generating an electron beam and an anode;
wherein, for moving the focal spot along the z-direction, the radiation source is adapted for deflecting the electron beam before it impinges on the anode.

7. The examination apparatus of claim 1,
wherein, for moving the focal spot along the z-direction, the anode is adapted as a rotating anode with a focal track;
wherein the rotating anode is adapted for rotating around a rotational axis; and
wherein the focal track has one of a sinusoidal form, a triangular form, and a saddle shaped form.

8. The examination apparatus of claim 1, further comprising:
a detector for a detection of electromagnetic radiation from the radiation source; and
a reconstruction unit for a reconstruction of an image of the object of interest on the basis of the detected radiation.

9. The examination apparatus of claim 8,
wherein the detector is adapted for moving along the z-direction in the opposite direction of the source.

10. The examination apparatus of claim 8,
wherein the detected radiation comprises oscillation data resulting from the back and forth movement of the focal spot and, in combination, circular data resulting from a circular trajectory of the focal spot.

11. The examination apparatus of claim 10,
wherein the oscillation data is detected during a first rotation of the radiation source; and
wherein the circular data is detected during a second rotation of the radiation source.

12. The examination apparatus of claim 10,
wherein the radiation source is adapted as a dual-tube system; and
wherein the oscillation data and the circular data are detected simultaneously.

13. The examination apparatus of claim 1, the examination apparatus being configured as one of the group consisting of a material testing apparatus, a medical application apparatus and a micro CT system.

14. The examination apparatus of claim 1, the examination apparatus being adapted as one of a 3D computed tomography apparatus and a 3D rotational X-ray apparatus.

15. The examination apparatus of claim 1,
wherein the electromagnetic radiation beam is a polychromatic x-ray beam.

16. An x-ray tube for an examination apparatus and adapted for emitting an electromagnetic radiation beam with a focal spot to an object of interest to be examined and for rotating around a z-axis around the object of interest with a rotational frequency;
wherein the x-ray tube is further adapted such that the focal spot of the beam moves back and forth along the z-direction of the z-axis with an oscillation frequency which is higher than the rotational frequency of the source.

17. A method of examination of an object of interest with an examination apparatus, method comprising the steps of:
emitting, by a radiation source, an electromagnetic radiation beam with a focal spot to the object of interest;
rotating of the source around a z-axis around the object of interest with a rotational frequency; and
moving of the focal spot of the beam back and forth along the z-direction of the z-axis with an oscillation frequency which is higher than the rotational frequency of the source.

18. (canceled)

19. (canceled)

20. The examination apparatus of claim 1, being adapted for cardiac CT.

21. The examination apparatus of claim 1,
wherein the trajectory is realized by a movement of the object in z-direction rather than by a movement of the radiation source.
